(19) United States
(12) Patent Application Publication
(10) Pub. No.: US 2026/0232421 A1
(43) Pub. Date: Aug. 13, 2026

(54) HANDS-FREE FLUSHING APPARATUS FOR DENTAL WATERLINES AND CONNECTED DENTAL DEVICES

(71) Applicant: A-dec, Inc., Newberg, OR (US)

(72) Inventors: Eric Walters, Tigard, OR (US); Jonathan Wilson, Beaverton, OR (US); Kohler Johnson, Dundee, OR (US)

(21) Appl. No.: 19/050,047

(22) Filed: Feb. 10, 2025

(51) Int. Cl. A61C 19/00

(52) U.S. Cl. CPC A61C 19/002

(57) ABSTRACT

A flushing device for dental waterlines comprises a holder having at least a first flush position, a second flush position, and a separating wall separating the first flush position and the second flush position. The first flush position is configured to receive and support a first dental tubing that includes a waterline, and the second flush position is configured to receive and support a second dental tubing that includes a waterline. The first and second flush positions are configured to hold the first and second dental tubings without contacting respective distal ends thereof. An optional receptacle that is configured to nest with the holder and support it from below can be used to position the holder at a desired height and to contain liquids used in flushing.

### BACKGROUND

Dental delivery systems provide pressurized water to various dental devices used to administer dental treatment to patients. Keeping dental delivery system waterlines free from biological contamination is an ongoing requirement. Frequent flushing of the waterlines is a recommended practice to reduce risk of infection because the relatively high-velocity flows produced in flushing procedures helps purge microorganisms that may have entered the waterlines during patient treatment and strip nascent growth from the inner walls of the waterlines. Flushing also introduces fresh water into the waterlines, including any water chemistry treatment additives that may be added to the water.

The waterlines are flushed while they remain housed in surrounding dental tubings. The dental tubings are connected at their proximal ends to the dental delivery system and have opposite, distal ends for connection to dental handpieces, dental syringes and other types of dental devices. According to one common configuration, the distal end of the dental tubing has a fitting, such as a nut, that is configured to be captive on the dental tubing and can form a threaded connection with the dental handpieces, dental syringes, quick disconnects and other dental devices. Dental tubings are sometimes referred to as hoses or cords.

Flushing of dental waterlines after every patient visit is a general recommendation and is a requirement in some jurisdictions. Conventional flushing procedures, however, are time consuming and can be difficult to complete effectively in the limited time available between consecutive patient visits. Flushing may be carried out in conjunction with an automated flushing mode running on the dental delivery system, which configures selected dental tubings for flushing, provides prompts to the user and/or provides flushing flows according to specific parameters (e.g., sequences, durations, contents, etc.).

Still, flushing procedures as currently carried out in many dental operatories require the user's concerted manual handling and constant attention. For example, current flushing practices may require that some dental tubings (e.g., for dental handpieces other than dental syringes) are flushed in one step of a sequence (with the handpieces removed, and with a nut, a quick disconnect or a motor in place at the ends thereof). Other dental tubings (e.g., for dental syringes) may then need to be flushed in a separate step (with the syringe bodies connected, syringe tips removed and while the user depresses the water button on each syringe body to keep water flowing through it during the step). The user may have to actuate certain momentary switches as well. During each flushing step, the user typically must hold the dental tubings being flushed over a sink or receptacle. Thus, the user typically needs more than their two hands to complete all steps, so the flushing is carried out in a sequence of two or more separate steps, which takes more time.

Some advanced dental delivery systems feature a cuspidor with an integrated cuspidor drain and a flushing apparatus (sometime referred to as a support center) configured for use with the cuspidor drain. These systems can be used with a program of automatic timed flush sequences. These systems have a flush container component that must be installed over the cuspidor like a cover and having receptacles to receive and hold dental tubings and, in some cases, a syringe body component (which remains connected to its dental tubing), during flushing. The Anthos R7 delivery system (available from Cefla s.c., Imola, Italy) has a flush container that fits over the cuspidor and has defined sockets for dental tubings and a dental syringe body. The dental syringe body must first be fitted with a separate adapter piece before it is inserted into the syringe body socket.

In the KaVo Estetica E50 Life/Primus 1058 delivery system, the flush container that fits over the cuspidor has spaces for two dental syringe bodies. When a syringe body is fit into its space, the water button on the syringe body is actuated by a spring-loaded pusher element. Each syringe body space is covered by a door that must be lifted to access the space and install or remove the syringe body. The flush container has grommets made of a flexible material in the receptacles for the dental tubings. The dental tubings are received by handpiece quick connect or electric motor connect fittings at the distal ends of the dental tubings.

Splatter from instrument flushing can contaminate the grommets (or other surfaces) that directly contact the dental tubings and/or their ends, which can result in contamination of the dental tubings. Additionally, when inserting or removing the dental tubings, which is carried out in a vertical direction, drops from the waterlines can land on the grommets and result in transfer of contaminants to any tubing surfaces that subsequently contact the grommets.

In the advanced dental delivery systems described above, multiple flush positions are defined for the dental tubings without the connected syringe bodies that have close-fitting receptacles which surround the dental tubings and their ends (handpiece nut alone or with attached quick disconnect and/or motor). In some cases, the defined flush positions may be sized and/or shaped to accommodate only a specific brand or line of dental tubings.

Even if a cuspidor is available, using the cuspidor system for flushing dental waterlines may be less preferred. For example, the amalgam separator typically integrated into the cuspidor system that traps amalgam waste prevents use of certain waterline infection control products that should not contact amalgam waste. Also, current flushing procedures carried out in a conventional flush container or support center positioned over a cuspidor do not allow for easy visualization of the flushing steps, including if color changes need to be monitored. For dental operatories without a cuspidor, flushing is typically carried out at a nearby sink with a drain or over a container, e.g., a bucket (which is later emptied into an appropriate a drain).

### SUMMARY

Described below are implementations of a flushing apparatus for dental waterlines that addresses shortcomings of currently used devices and methods.

According to a first implementation, a flushing device for dental waterlines, comprises a holder having at least a first flush position, a second flush position, and a separating wall separating the first and second flush positions. The first flush position is configured to receive and support a first dental tubing that includes a waterline, and the second flush position is configured to receive and support a second dental tubing that includes a waterline. The first and second flush positions are configured to hold the first and second dental tubings without contacting respective distal ends thereof.

The first and second flush positions can each comprise a respective dental tubing rest and a pair of arms. The first and second flush positions can be configured to hold the respective first and second dental tubings in generally horizontal positions with the distal ends thereof cantilevered over a sink or a receptacle. The first and second flush positions can be configured to cradle the respective first and second dental tubings such that distal ends thereof are suspended.

The first and second flush positions can comprise tapered surfaces configured to contact corresponding tapered surfaces of the respective first and second dental tubings at point spaced proximally from the distal ends thereof. The tapered surfaces of the first and second flush positions can be shaped to contact tapered outer surfaces of nuts on the first and second dental tubings.

The instrument holder can comprise downwardly extending legs configured to contact a base of a sink.

In the flushing device, an insertion direction for at least one of the first and second flush positions can be defined to extend distally, and a securing direction can be defined to extend proximally and substantially opposite the insertion direction. A dental tubing to be flushed can inserted distally in the insertion direction and then withdrawn proximally in the securing direction to secure the dental tubing in a secured position relative to the flushing device.

The flushing device may comprise at least one additional flush position defining a generally vertical insertion direction for inserting a respective dental instrument or portion thereof into the flushing device.

The flushing device may comprise at least one additional flush position comprising a recess with a protrusion shaped to engage a depressible control element on a respective dental instrument received in the recess.

The flushing device may comprise a receptacle. The receptacle and the instrument holder may be configured in a nesting arrangement relative to each other, with the receptacle being configured to support the instrument holder at a desired height and to contain fluids used in a flushing procedure. The receptacle may include upstanding walls and at least one handle by which a user can manipulate and/or carry the receptacle.

The first and second flush positions can be configured to cradle the respective first and second dental tubings such that distal ends of the first and second dental tubings are free from surrounding structure.

The first and second flush positions can comprise tapered surfaces configured to contact corresponding tapered surfaces of the first and second dental tubings. The tapered surfaces of the first and second flush positions can be shaped to contact tapered outer surfaces of nuts on the first and second dental tubings.

The instrument holder can comprise downwardly extending legs configured to contact a base of a sink.

In the flushing device, at least one of the first and second flush positions can be configured to define a generally horizontal insertion direction for inserting a respective dental tubing into the flushing device. Additionally or alternatively, at least one of the first and second flush positions can be configured to define a generally vertical insertion direction for inserting a respective dental instrument or portion thereof into the flushing device.

At least one of the first and second flush positions can comprise a recess with a protrusion shaped to engage a depressible control element on a respective dental instrument received in the recess.

The flushing device can comprise an optional receptacle, wherein the receptacle and the instrument holder are configured in a nesting arrangement relative to each other, and wherein the receptacle is configured to support the instrument holder at a desired height and to contain fluids used in a flushing procedure. The receptacle can include upstanding walls and at least one overflow opening positioned to cause fluid in the receptacle to overflow out of the receptacle upon reaching a predetermined height.

According to another implementation, a flushing device for dental waterlines, comprises an instrument holder having a body with a top, sides extending downwardly from the top, and at least first and second flush positions defined in the body and a receptacle positionable below the instrument holder. The first flush position is configured to receive a first dental tubing that includes a waterline, and the second flush position is configured to receive a second dental tubing that includes a waterline. The receptacle is engageable with the instrument holder to contain waste liquids from flushing. The receptacle and the instrument holder are movable, and the instrument holder is separable from the receptacle for use in a freestanding configuration.

The first flush position can define a generally horizontal insertion axis for the first dental tubing, and wherein the first flush position is configured to cradle the first dental tubing with a distal end of the first dental tubing cantilevered over the receptacle. The first flush position can be configured to support a tapered surface of the first dental tubing without contacting a distal end of the first dental tubing. For example, the first flush position can be configured to contact and support a tapered handpiece nut of the first dental tubing. The first flush position can be sized to accommodate a dental tubing with a connected device or a portion thereof extending distally of the tapered handpiece nut.

The second flush position can define a generally vertical insertion axis for the second dental tubing, and the second flush position can comprise a closely-fitting recess configured to receive a dental syringe body connected to the second dental tubing. The second flush position can comprise a protrusion in the recess configured to depress a water button of the dental syringe body received in the second flush position.

The instrument holder and the receptacle can be engageable with each other in a nested arrangement.

The foregoing and other objects, features, and advantages will become more apparent from the following detailed description, which proceeds with reference to the accompanying figures.

### BRIEF DESCRIPTION OF THE DRAWINGS

FIG. 1 is a perspective view of a flushing device according to a first implementation.

FIG. 2 is a front elevation view of the flushing device of FIG. 1.

FIG. 3 is a right side elevation view of the flushing device of FIG. 1

FIG. 4 is a left side elevation view of the flushing device of FIG. 1.

FIG. 5 is a rear elevation view of the flushing device of FIG. 1.

FIG. 6 is a top plan view of the flushing device of FIG. 1.

FIG. 7 is a bottom plan view of the flushing device of FIG. 1.

FIG. 8 is another perspective view of the flushing device showing the holder spaced above the receptacle.

FIG. 9 is a top plan view of the receptacle.

FIG. 10 is a perspective view of the receptacle.

FIG. 11 is a bottom plan view of the holder.

FIG. 12 is a perspective view of the holder.

FIG. 13 is an enlarged view of the flush positions in the top of the flushing device.

FIG. 14 is an enlarged view of the flush positions in the front side of the holder.

FIG. 15 is a perspective view of the flushing device with dental tubings of different configurations received in the flush positions in the top and in the front side.

FIG. 16 is a front elevation view of the flushing device with dental tubings of FIG. 15.

FIG. 17 is a left side elevation view of the flushing device with the dental tubings of FIG. 15.

FIG. 18 is a top plan view of the flushing device with dental tubings of FIG. 15.

FIG. 19 is an assembly view showing four dental tubings of two different end configurations being inserted into respective front flush positions and three dental tubings with instruments (syringe bodies) being inserted into respective top flush positions.

FIG. 20 is a section view in elevation showing the configuration of the top flush positions.

FIG. 21 is another section view in elevation taken along the line 21 -21 in FIG. 18 and showing a dental tubing with a handpiece motor received in a front flush position and a syringe body received in a top flush position.

### DETAILED DESCRIPTION

Described below are implementations of a flushing apparatus for flushing dental tubings and more specifically, dental waterlines running within the dental tubings. Dental waterlines supply water (and in some cases, other liquids) to dental devices. Dental devices as used in this context include dental handpieces and dental air/water syringes that are connected to respective dental tubings (and waterlines) to enable normal operation. In addition to flushing the dental waterlines while housed in the dental tubings, the flushing apparatus is also preferably configured to flush certain types of dental devices that may remain connected to some the dental tubings. The set of dental tubings without connected dental devices and the dental tubings with connected dental devices (e.g., syringe bodies) to be flushed is sometimes referred to herein as the “flush targets.”

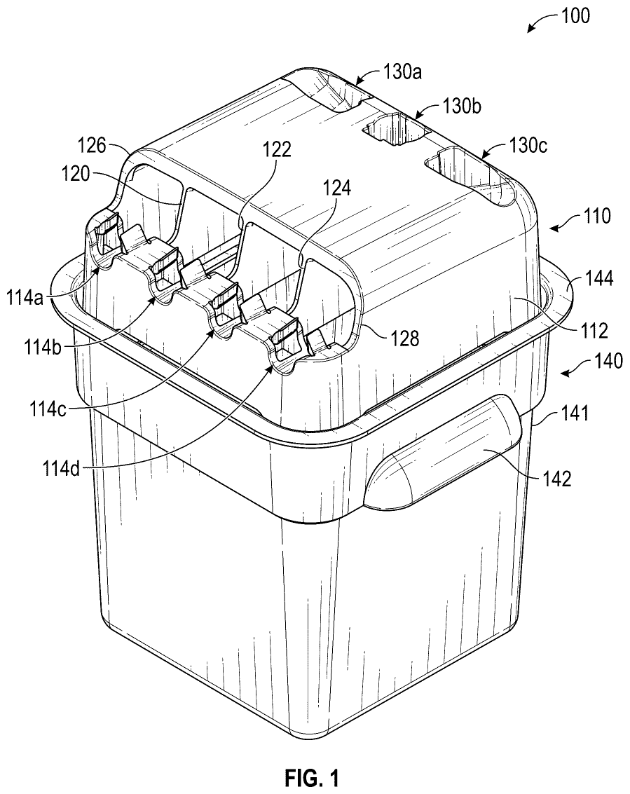

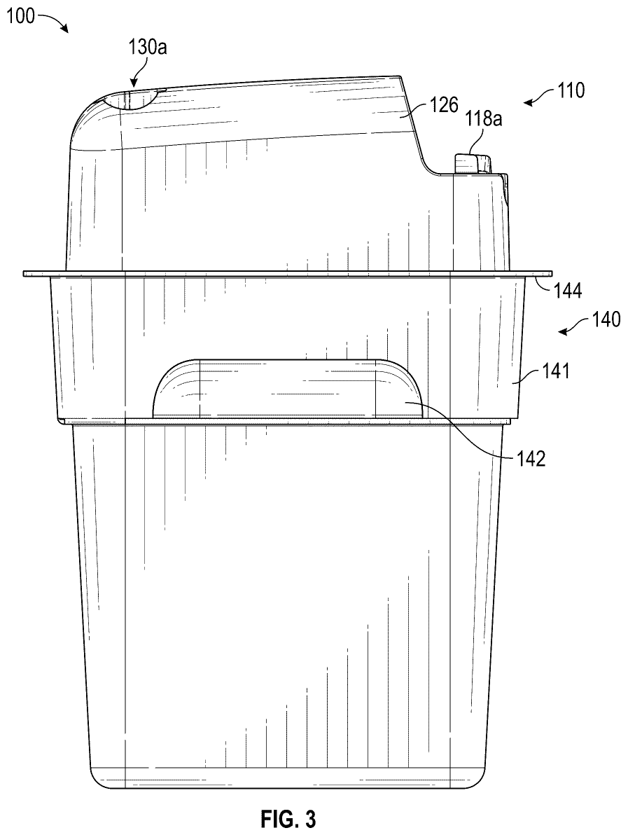

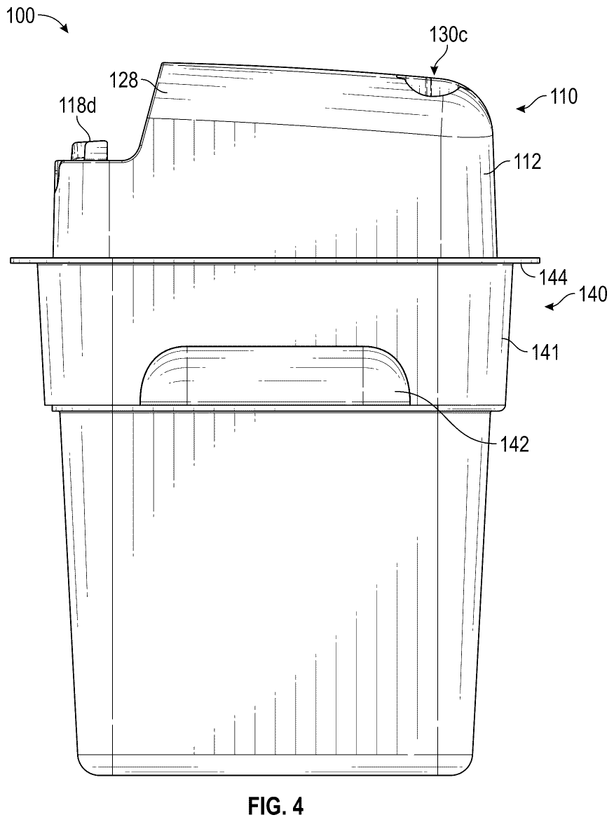

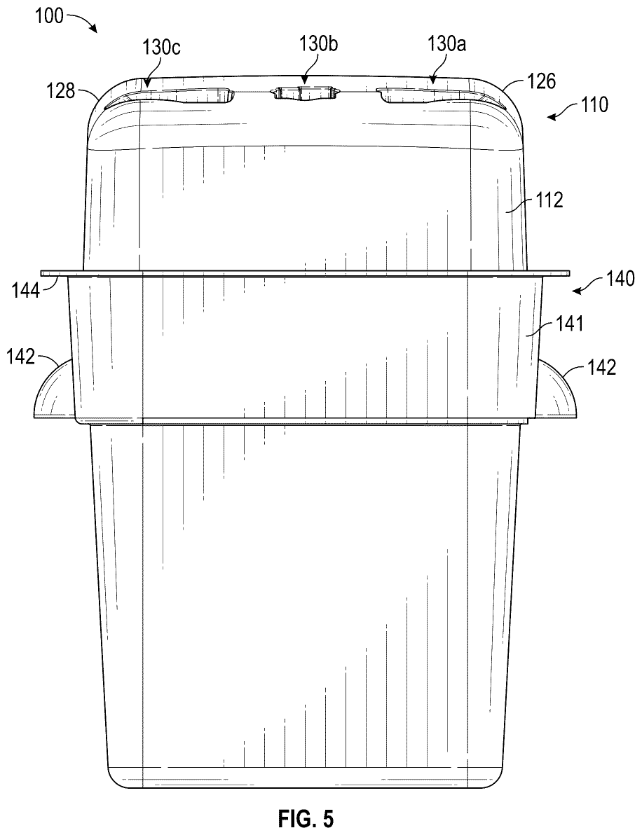

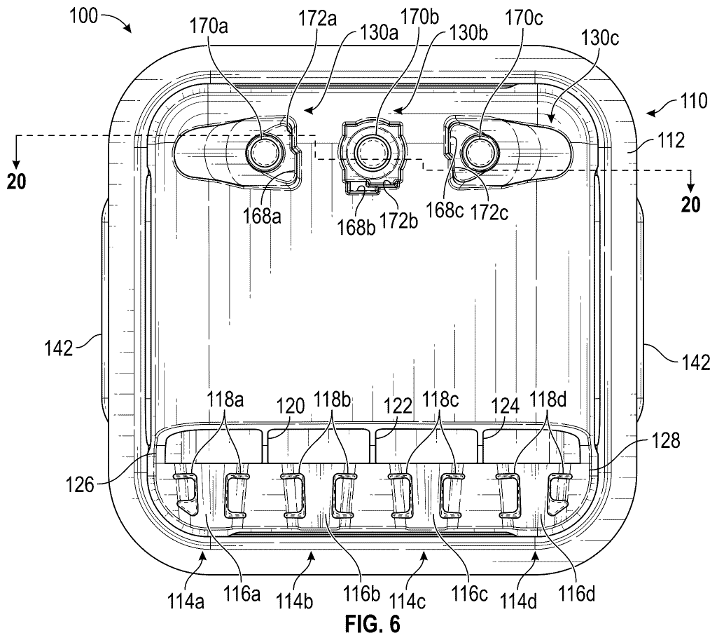

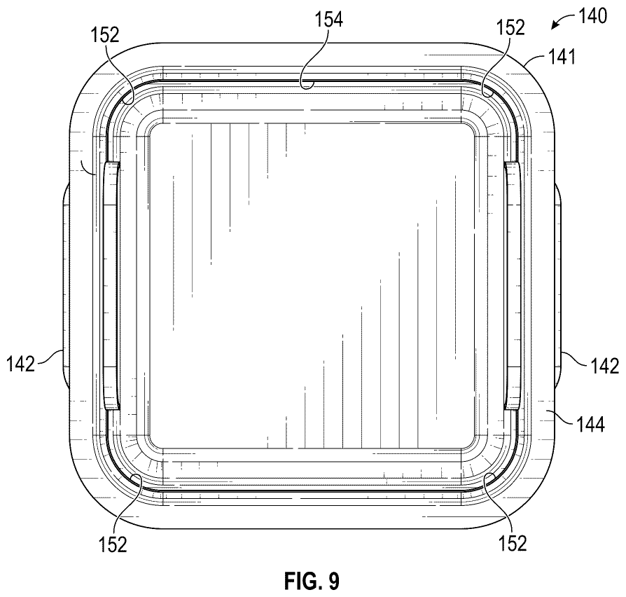

Referring first to FIGS. 1-9, according to a first implementation, a flushing device 100 for flushing dental tubings has a first portion or component for holding dental tubings (or select dental devices connected to dental tubings), referred to as a holder 110, and an optional support or receptacle 140. The receptacle 140 can be assembled as shown to support the holder 110 from below to position the holder 110 at a desired height and/or receive and contain liquid(s) used in the flushing process, as is described below in more detail.

The holder 110 has a body 112 in which multiple flush positions are defined. In the illustrated example, there are seven total flush positions, including four flush positions 114a, 114b, 114c and 114d defined at the front side and top of the instrument holder 110 (also sometimes referred to as “front flush positions”), and three flush positions 130a, 130b and 130c defined in the top of instrument holder (also sometimes referred to as “top flush positions”). In other examples, there can be any number front flush positions and/or top flush positions for a total number of flush positions of one or more. In addition, there can be flush positions that are defined in different surfaces than the illustrated front flush positions and top flush positions.

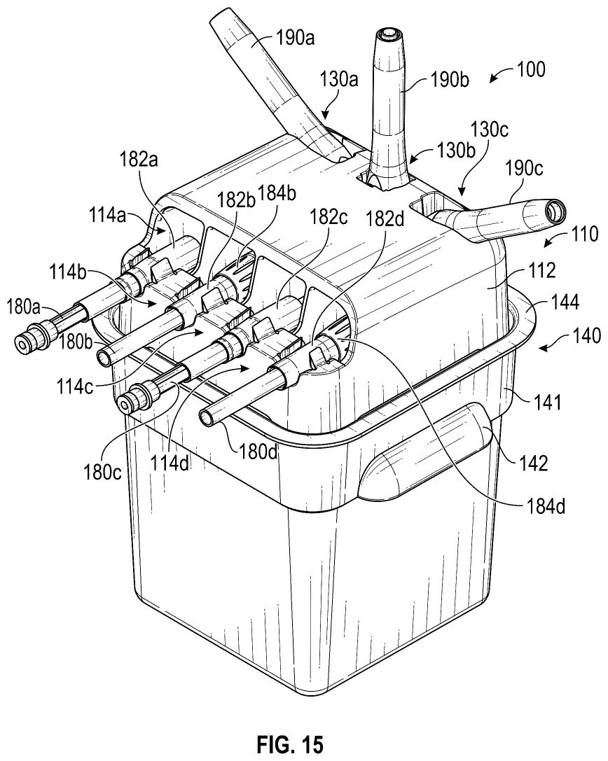

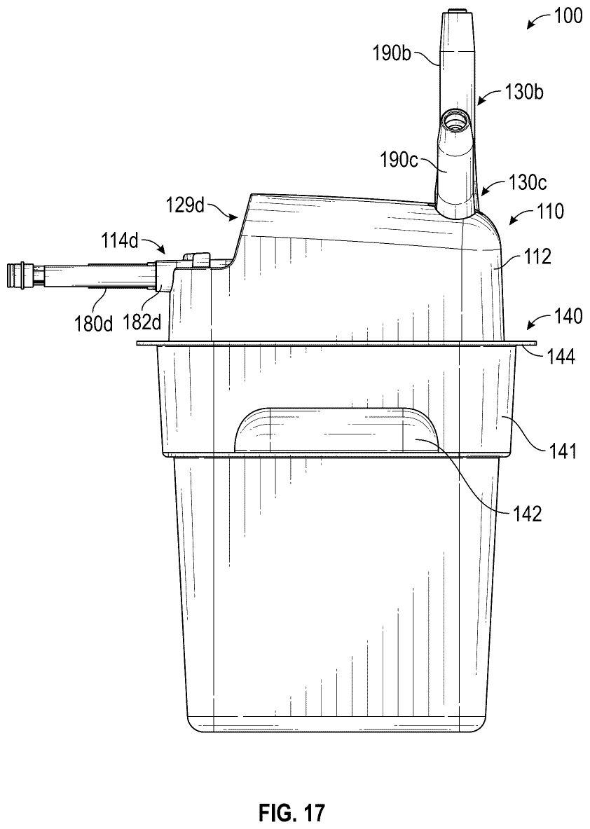

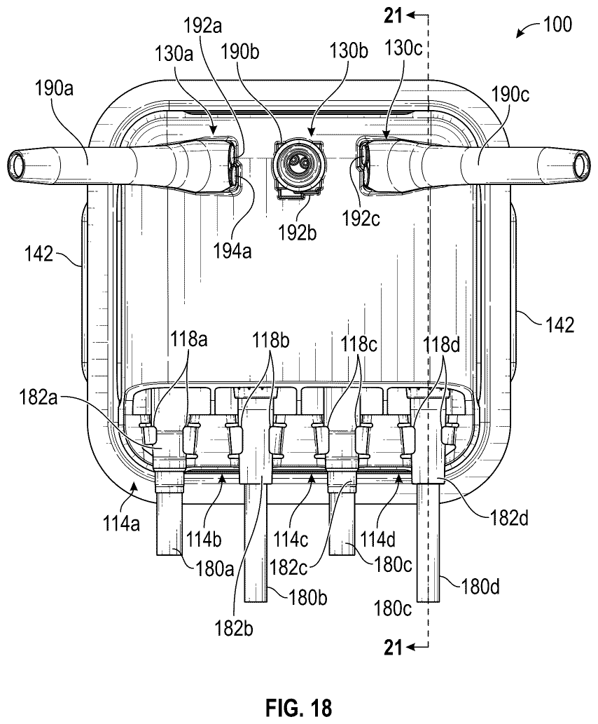

The front flush positions 114a-114d are each configured to hold a dental tubing in a preferred orientation for flushing, such as a generally horizontal orientation for the implementation of FIGS. 1-9. FIGS. 15-19 show the flushing device 100 with respective dental tubings 180a, 180b, 180c and 180d held generally horizontally in the front flush positions 114a-114d, respectively. As described below in more detail, the flush positions 114a-114d have respective insertion axes along which tubings are installed and removed that are horizontal or slightly inclined relative to horizontal (referred to herein as generally horizontal).

As described in more detail below, the front flush positions 114a-114d are configured to hold the dental tubings 180a-180d spaced apart from each other and at a height above a level to which liquids used in the flushing process may accumulate (either in the receptacle 140 or around the instrument holder 110 if used on its own in a sink).

It is noted that the dental tubings 180a-180d, as well as the syringe bodies 190a, 190b and 190c, are shown in FIGS. 15-19 with abbreviated lengths for clarity of illustration. Typically, each dental tubing would extend over a length of at least several feet to a connection at its proximal end (not shown) to a control head or other apparatus. Among other functions, the control head feeds water and other liquids through the waterlines for normal operations and flushing operations. For flushing operations, the liquids fed through the waterlines are water, solutions and/or waterline cleaning products.

Flushing procedures for dental waterlines can include flushing carried out between patients as well flushing undertaken other intervals, such as the end of each workday, each work week, etc. Flushing can be accomplished with water (from a water mains supply or a bottled dental water supply), and may include infection control chemistry products. Two types of infection control chemistry products are sold by the present assignee under the tradenames ICX and ICX Renew.

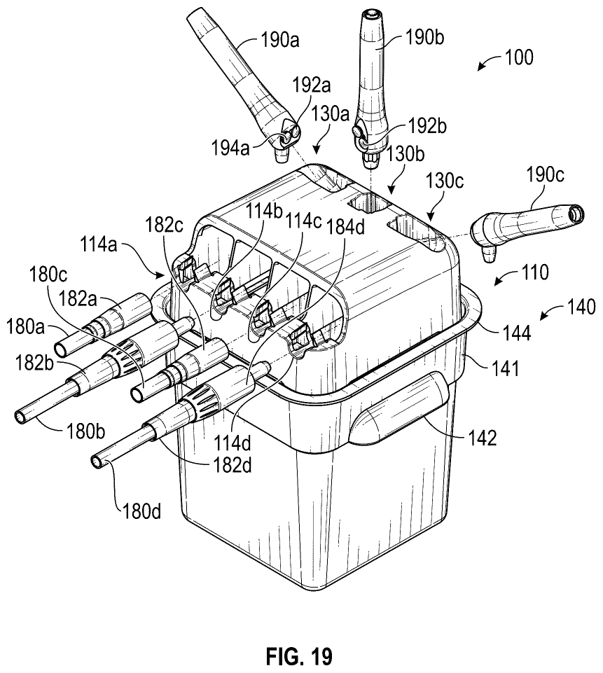

Referring to FIGS. 15 and 19, the dental tubings 180a-180d of the illustrated example are shown with two different end configurations. For the dental tubings 180a and 180c, each has an end defined by a respective nut (or handpiece nut) 182a, 182c. The nuts 182a, 182b are typically configured as captive nuts that remain on the dental tubings 180a, 180c, and thus can be considered components of the tubings. For the dental tubings 180b and 180d, each also has a nut 182b, 182d, respectively, but each end is defined by a connected handpiece motor 184b, 184d, respectively. The handpiece motors 184b, 184d are components of handpieces, i.e., which are types of connected dental devices. The handpiece motors 184b, 184d are electric handpiece motors that are typically not expected to be flushed, but may be flushed incidentally if they remain connected. In the same way, other components, such as, e.g., handpieces and/or quick disconnects could also be connected to the tubings/nuts and thus present for flushing in some scenarios, although such components are not typically targeted for flushing.

As shown in FIGS. 1, 2, 15, 17 and 18, the front flush positions 114a-114d are configured such that each dental tubing 180a-180d, when held in position, contacts the receptacle 140 only at an intermediate point spaced from its distal end, while the distal end is cantilevered over the open interior area of the receptacle 140. The distal ends may be handpiece nuts, quick disconnects and/or motors, all of which typically have a seal. Thus, the distal ends, and particularly the respective seals, are not contacted by any structure, which reduces the chances of cross-contamination. In other conventional flushing devices, there is a chance of cross contamination because the distal end, and particularly the seal thereof, is allowed to contact the structure in which the distal end is received, which could allow for spread of infection from a different distal end that contacted the same location in a prior flushing session. Also, because the distal ends in the flush positions 114a-114d are not obstructed by closely-fitting structure(s), the distal ends can be more effectively cleaned during flushing.

The configuration of the front flush positions 114a-114d can also be described is alternatively described as providing “cradling flush positions” that cradle the dental tubings. In this way, flushing can be carried out effectively with the spent flushing liquids draining freely from the distal ends into the receptacle 140 below.

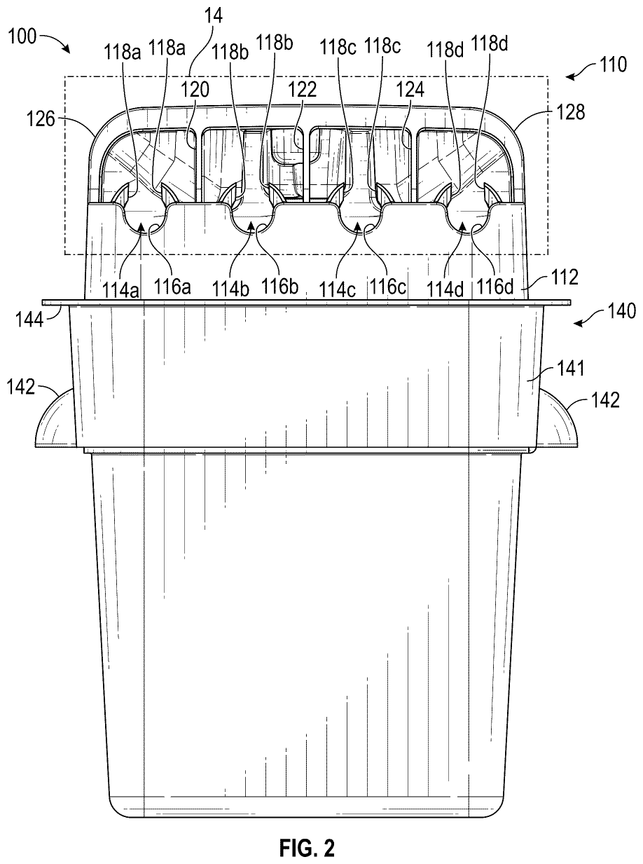

Referring to FIG. 2, the front flush positions 114a-114d can have respective surfaces that support an end of the dental tubing (such as the nut), which are referred to as end rests 116a, 116b, 116c and 116d. The front flush positions 114a-114d may also include respective pairs of arms 118a, 118b, 118c and 118d, which, together with the end rests 116a-116d, allow the respective dental tubings 180a-180d to be easily inserted, securely held (or cradled) and easily released.

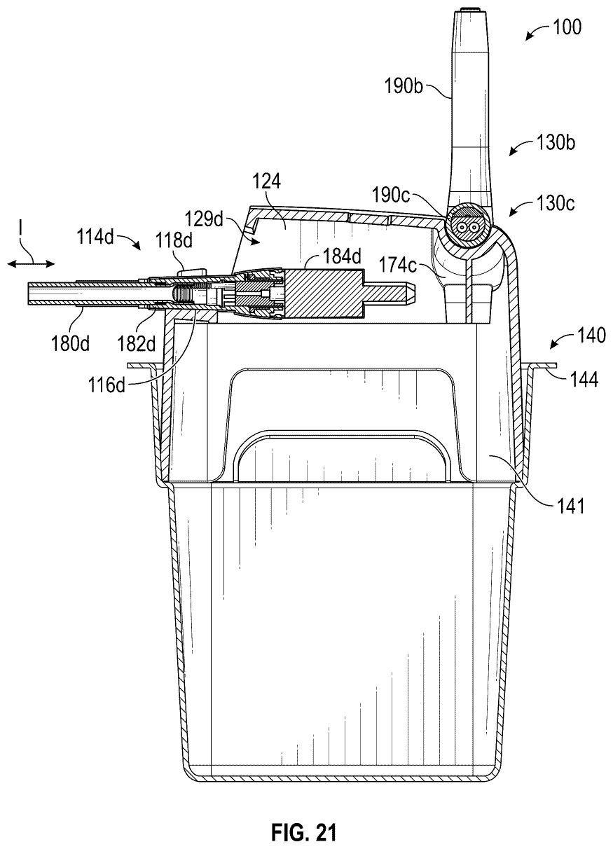

In the illustrated implementations, the end rests 116a-116d and the pairs of arms 118a-118d optionally define respective openings that taper to receive the tapered outer surfaces of the nuts 182a-182d. Thus, to place the dental tubing 180d in the received position as shown in FIGS. 15 and 21, the tubing 180 d is first advanced (distally) along an insertion axis (arrow I in FIG. 21) into the instrument holder 110 above the flush position 114d until the tubing 180d can be inserted downwardly between the pair of arms 118d. Next, the tubing 180d is pulled rearwardly (proximally) to engage the nut 182d with the end rest 116d below and the pair of arms 118d above, securing the tubing 180d in place and cradling it relative to the instrument holder 110. The tubing 180d is held in place by the contact between the tapered outer surface of the nut 182d and the corresponding tapered surface(s) of the end rest 116d and the pair of arms 118d. As can be seen in FIG. 21, the dental tubing 180d is held in position only at a point spaced from its distal end. Thus, the distal end (in this case, an end of the motor 184d, but it could be a handpiece nut or a quick disconnect) is not in contact with or close to any surface of the holder 110 or the receptacle 140 that is likely to be contacted with liquid during the flushing process. The dental tubing 180d is cantilevered over the receptacle 140, and spent flushing liquid can freely drain into the receptacle. Meanwhile, because of its positioning in the holder 110, the dental tubing 180 can be viewed by the user so that they can verify that flushing operations are taking place correctly.

In the illustrated implementations, the holder 110 includes optional structures that at least partially separate one or more of the flush positions 114a, 114b, 114c and 114d from adjacent flush positions. For example, referring to FIGS. 1 and 2, the holder 110 can have separating walls 120, 122 and/or 124. The separating wall 120 at least partially separates the flush position 114a from the flush position 114b. Similarly, the separating wall 122 separates the flush position 114b from the flush position 114c. In the same way, the separating wall 124 separates the flush position 114c from the flush position 114d. The separating walls 120, 122, 124 help to reduce spray, droplets and other forms of liquid from one waterline (or flush position) from entering into contact with another instrument (or flush position), which reduces the potential for cross-contamination. The separating walls 120, 122, 124, end walls 126 and 128, and the adjoining top surface, together define respective alcoves 129a, 129b, 129c and 129d associated with the front flush positions 114a-114d.

The top flush positions 130a, 130b and 130c are each configured to hold a connected dental device or portion thereof in a preferred orientation for flushing, such as a generally vertical orientation for the implementation of FIGS. 1-9, while each dental device remains connected to its respective tubing (and waterline). Also, the top flush positions 130a, 130b and 130c are specially configured to receive dental devices of a certain type(s) and, optionally, to interact with a control element of the dental devices. For example, the top flush positions 130a, 130b and 130c may be shaped to receive dental syringes, and specifically, respective syringe bodies 190a, 190b and 190c, as shown in FIG. 19. As shown in FIG. 19, the syringe bodies 190a-190c have been prepared for flushing by having their syringe tips removed (the syringe tips are separately sterilized or replaced). The dental syringe bodies 190a and 190c define a form factor sometimes referred to as an angled syringe. The syringe body 190b defines a form factor sometimes referred to as a pencil or straight syringe.

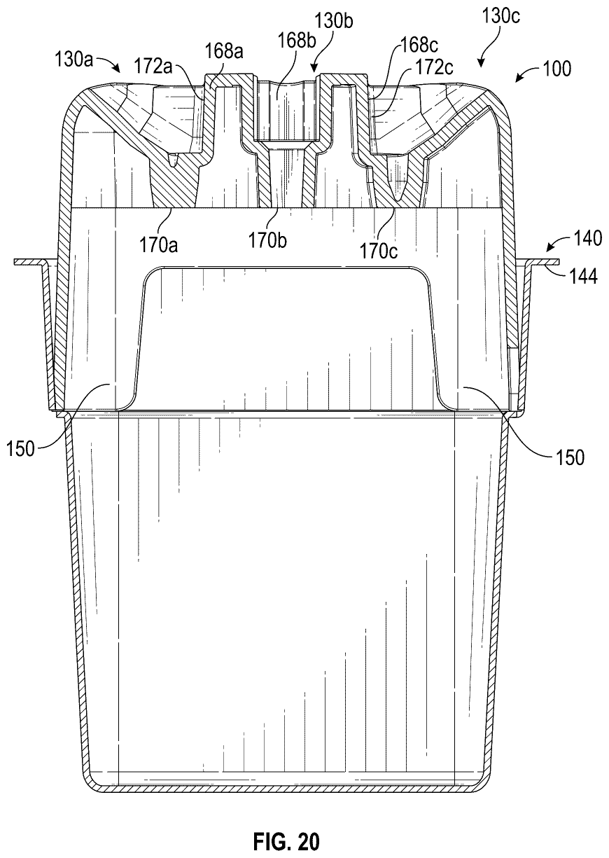

As shown, at least a distal portion of each of the syringe bodies 190a-190c is oriented generally vertically. The top flush positions 130a-130c have socket-like shapes or recesses that closely follow the outer contours of the received portions of the syringe bodies 190a-190c, respectively. The top flush positions 130a-130c have respective insertion axes that are vertical or slightly inclined relative to vertical (referred to herein as “generally vertical”). Respective openings 170a, 170b and 170c (FIG. 20) in the flush positions 130a, 130b and 130c allow liquid from the syringe bodies 190a, 190b and 190c to drain to the receptacle 140, if arranged as in FIGS. 1-9, or a sink or other vessel, if provided below.

The flush position 130a for the dental syringe 190a can be configured with a recess wall 168a that has a protrusion 172a shaped to engage (e.g., depress) a water button 192a on the syringe body 190a when the syringe body 190a in inserted into the flush position 130a. The air button 194a, however, is spaced apart from and does not contact the recess wall 168a. In this way, the user can configure the syringe body 190a so that the water button stays depressed during flushing without needing to manually actuate it. Similarly, the flush position 130b has a recess wall 168b with a protrusion 172b shaped to depress a water button 192b on the syringe body 190b. In the same way, the flush position 130c has a recess wall 168c with a protrusion 172c shaped to depress a water button 192c on the syringe body 190c.

The various recess walls of the flush positions 130a-130c tend to keep each of these positions (and any dental device received therein) isolated from the others, which helps reduce the chance of cross-contamination. In addition, inner recess walls 174a, 174b and 174c (FIG. 13) tend to separate the flush positions 130a-c from the flush positions 114a-114d.

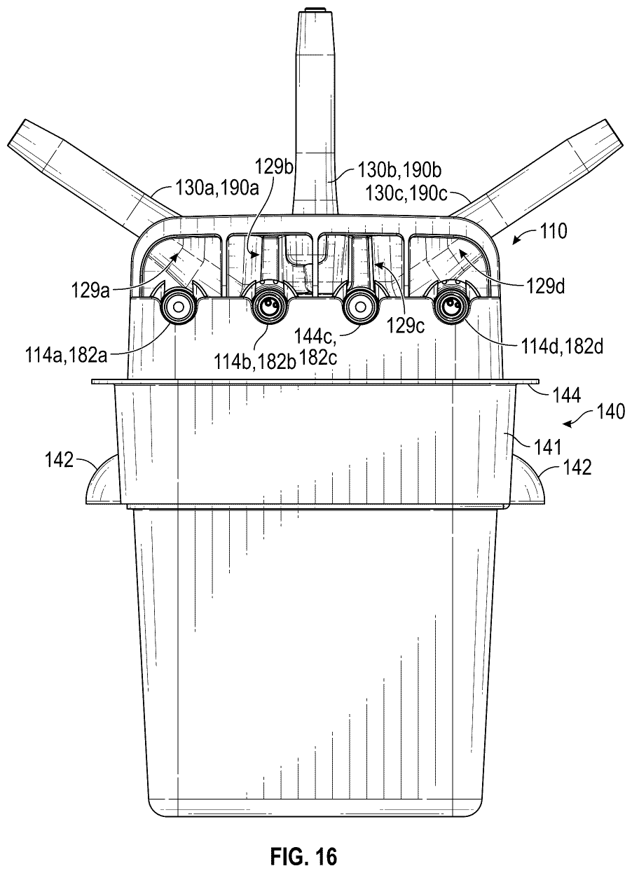

As shown in FIGS. 15-18, after the dental tubings 180a-180d have been inserted into the flush positions 114a-114d and within their alcoves 129a-129d (FIG. 16), and the syringe bodies 190a-190c have been inserted into the flush positions 130a-130c, the flushing device 100 is now configured for hands-free flushing. Flushing can commence, either by being manually initiated by the user or via a timed flushing feature that may be run on the delivery system. For example, the A-dec 300 Pro and A-dec 500 Pro delivery systems provide timed flushing procedures with on-screen guidance to the user. Further details about automated flushing procedures are described in U.S. Pat. No. 12,097,084, which is incorporated herein by reference.

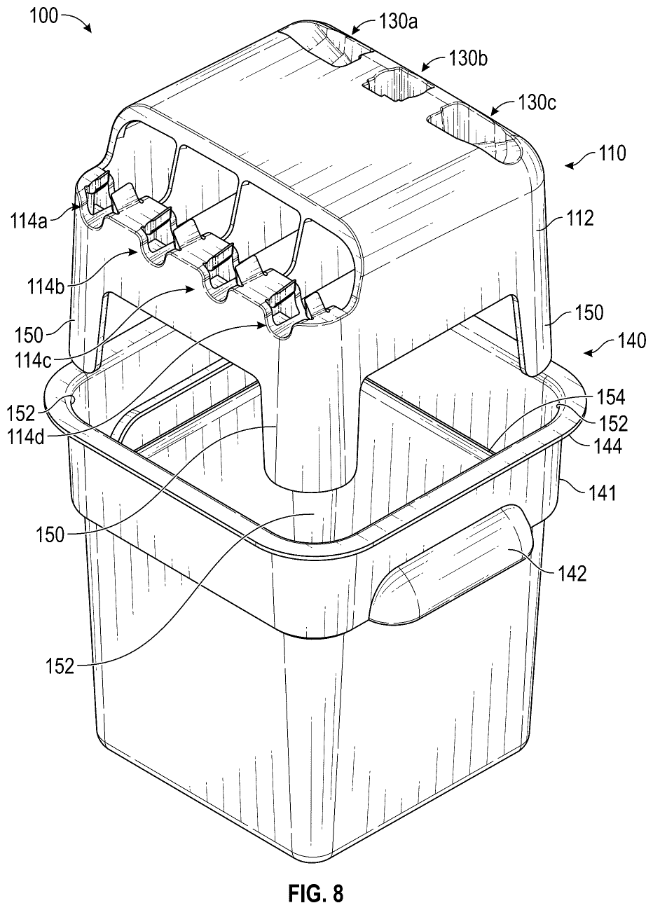

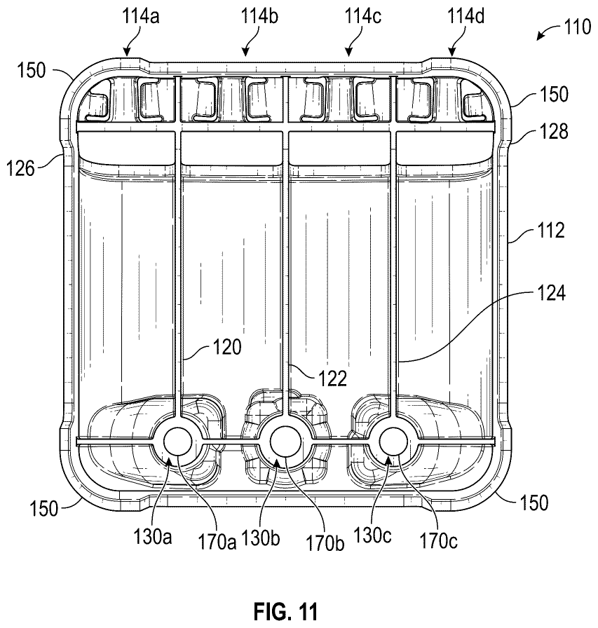

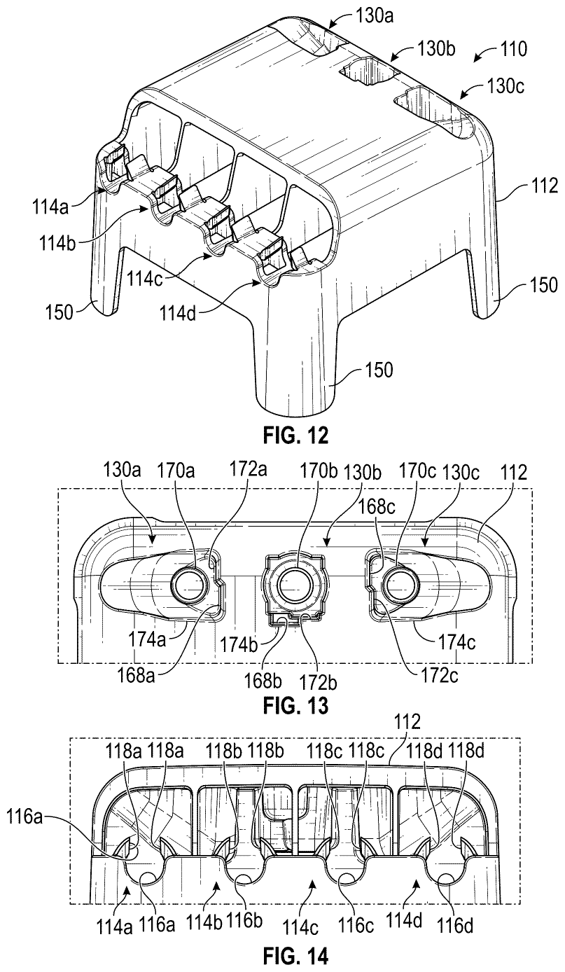

In FIGS. 1-9, 11 and 12, the instrument holder 110 has a rectangular (square) shape with rounded corners when viewed from above (i.e., in plan), but other shapes are also possible. As best shown in FIG. 8, the instrument holder 110 can have a freestanding configuration (i.e., such that it is self-supported), e.g., with a base or legs 150 that are configured so that the flush positions and/or overall height are as desired.

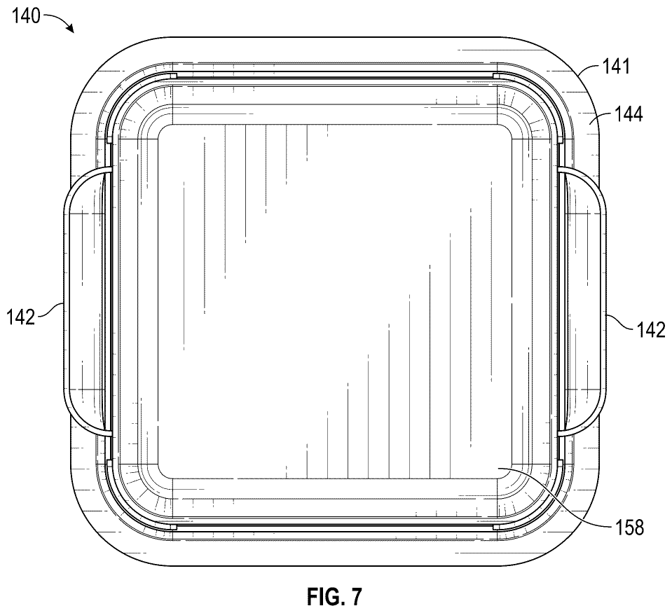

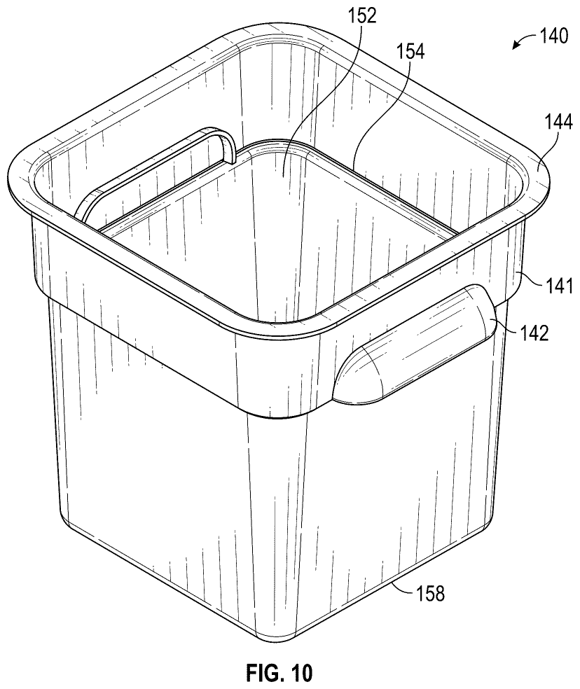

The receptacle 140 can have a body 141 with an open upper end and a flange 144. The receptacle typically has a closed bottom 158 (FIG. 7) as shown. The receptacle 140 can be configured to support the instrument holder 110 in a desired position(s), such as a nested arrangement as shown. For example, as shown in FIGS. 8-10, the receptacle 140 can have corners 152 and a rib 154 upon which the legs 150 of the instrument holder 110 can engage/rest on in an inter-fitted arrangement.

One or more carrying handles 142 can be provided on the receptacle 140 to allow the user to easily maneuver and carry the flushing device 100. In the illustrated implementation, the handles 142 are formed in the body 141 at a height generally above the rib 154 and below the flange 144.

The receptacle 140 and the instrument holder 110 are designed such that the instrument holder 110 nests within the open top of the receptacle 140. Other inter-fitted arrangements between the receptacle 140 and the instrument holder 110 are also possible. In case liquid is allowed to fill the receptacle 140 completely, it then overflows over the upper edge of the receptacle 140. Meanwhile, the flush positions 114a-114d and 130a-130c and the dental tubings 180a-180d and syringe bodies 190a-190c in the instrument holder 110 remain above the overflowing liquid, thus reducing the risk of cross-contamination. In some implementations, the flush positions 114a-114d and 130a-130c are at least about one inch above the upper edge of the receptacle 140.

In some implementations, the receptacle 140 is formed of a material that is at least partially transparent or translucent to allow a user to visualize the level of liquid in the container. In some implementations, the receptacle 140 and the instrument holder 110 are formed with a minimum number of components (or even as one-piece constructions) to allow for more efficient manufacturing (e.g., by molding) and easier cleaning.

In some implementations, the receptacle 140 is designed to contain a desired liquid volume below the height of the flush positions. In some implementations, the desired liquid volume below the flush positions is about 2 liters. This desired liquid volume can be selected according to the maximum volume of the on-board water bottle system, as well as other considerations. In addition, the receptacle 140 can be provided with additional head room in excess of the desired liquid volume. For example, the desired liquid volume in the illustrated implementation is approximately 2 liters, and additional headroom of about 2 liters can be provided to increase the total volume to about 4 liters. In addition, the flush positions are positioned above the top edge of the receptacle (such as by about one inch in the illustrated implementations).

In some scenarios, the instrument holder 110 is used for flushing without the receptacle 140. For example, the instrument holder 110 may be placed in, on or over a sink such that liquids used in flushing drain to the sink drain. In some of these scenarios, the instrument holder is freestanding. It would also of course be possible to use the instrument holder 110 together with a container other than the receptacle 140.

Although it may be possible in some scenarios to arrange the instrument holder 110 to drain to the cuspidor drain, such a use is generally less preferred. According to a preferred scenario for the instrument holder 110 together with the receptacle 140, waste from flushing is captured in the receptacle 140, which in turn is portable and can be safely discarded in an approved manner, such as into an approved drain.

In addition, because the flushing device 100 is portable even when filled with waste liquids after a flushing procedure, one flushing device can be used with multiple delivery systems if convenient, which reduces the need to outfit each delivery system with a dedicated flushing waste container.

Alternatively, the instrument holder 110 and/or the receptacle 140 may be used in connection with a wall mount, a cabinet mount or other storage area, probably in or very near the dental operatory, with a dedicated drain. Such an area can also be used to store the flushing device 100 when not in use.

As set forth above, the flushing device 100 enables hands-free flushing (or at least easier flushing), which increases operatory turnover efficiency by reducing the steps and time needed for a user to complete a flushing sequence. Syringe water buttons are automatically actuated, such as by an integral feature of the holder 110 that depresses the water buttons. Other dental tubings are cradled in the holder 110 in designated flush positions with geometry that resembles the handpiece sockets (holders) of the delivery system. In this way, the flush positions are compatible with the dental tubings of most dental devices. Also, because the dental tubings are cradled, their touch points with other structures (particularly near their wet ends) and the associated cross-contamination risks are reduced.

The examples and embodiments described herein are to be considered in all respects as illustrative and not restrictive. The terms “first,” “second,” “top,” “bottom,” etc., as used herein, are intended for illustrative purposes only and do not limit the embodiments in any way. In particular, these terms do not imply any order or position of the components modified by such terms. Additionally, the term “plurality,” as used herein, indicates any number greater than one, either disjunctively or conjunctively, as necessary, up to an infinite number. Further, as used herein, “horizontal” and “vertical” are general relative terms. The definition of “vertical” is not limited to structures that are precisely perpendicular to the ground, the definition of “horizontal” is not limited to structures that are precisely parallel to the ground, and reference to different components as being “horizontal” and “vertical” does not imply that these components are precisely perpendicular to each other. Accordingly, while specific embodiments have been illustrated and described, numerous modifications come to mind without significantly departing from the spirit thereof.

In view of the many possible embodiments to which the disclosed principles may be applied, it should be recognized that the illustrated embodiments are only preferred examples and should not be taken as limiting in scope. Rather, the scope of protection is defined by the following claims. We therefore claim all that comes within the scope and spirit of these claims.

We claim:

1. A flushing device for dental waterlines, comprising:
a holder having at least a first flush position, a second flush position, and a separating wall separating the first and second flush positions,
wherein the first flush position is configured to receive and support a first dental tubing that includes a waterline, and the second flush position is configured to receive and support a second dental tubing that includes a waterline, and
wherein the first and second flush positions are configured to hold the first and second dental tubings without contacting respective distal ends thereof.

2. The flushing device of claim 1, wherein the first and second flush positions each comprise a respective dental tubing rest and pair of arms.

3. The flushing device of claim 1, wherein the first and second flush positions are configured to hold the respective first and second dental tubings in generally horizontal positions with the distal ends thereof cantilevered over a sink or a receptacle.

4. The flushing device of claim 1, wherein the first and second flush positions are configured to cradle the respective first and second dental tubings such that distal ends thereof are suspended.

5. The flushing device of claim 1, wherein the first and second flush positions comprise tapered surfaces configured to contact corresponding tapered surfaces of the respective first and second dental tubings at point spaced proximally from the distal ends thereof.

6. The flushing device of claim 5, wherein the tapered surfaces of the first and second flush positions are shaped to contact tapered outer surfaces of nuts on the first and second dental tubings.

7. The flushing device of claim 1, wherein the instrument holder comprises downwardly-extending legs configured to contact a base of a sink.

8. The flushing device of claim 1, wherein an insertion direction is defined for at least one of the first and second flush positions to extend distally, and wherein a securing direction is defined to extend proximally and substantially opposite the insertion direction, wherein a dental tubing to be flushed is inserted distally in the insertion direction and then withdrawn proximally in the securing direction to secure the dental tubing in a secured position relative to the flushing device.

9. The flushing device of claim 1, further comprising at least one additional flush position defining a generally vertical insertion direction for inserting a respective dental instrument or portion thereof into the flushing device.

10. The flushing device of claim 1, further comprising at least one additional flush position comprising a recess with a protrusion shaped to engage a depressible control element on a respective dental instrument received in the recess.

11. The flushing device of claim 1, further comprising a receptacle, wherein the receptacle and the instrument holder are configured in a nesting arrangement relative to each other, and wherein the receptacle is configured to support the instrument holder at a desired height and to contain fluids used in a flushing procedure.

12. The flushing device of claim 11, wherein the receptacle includes upstanding walls and at least one handle by which a user can manipulate and/or carry the receptacle.

13. A flushing device for dental waterlines, comprising:
an instrument holder having a body with a top, sides extending downwardly from the top, and at least first and second flush positions defined in the body; and
a receptacle positionable below and engageable with the instrument holder to contain waste liquids from flushing;
wherein the first flush position is configured to receive a first dental tubing that includes a waterline, and the second flush position is configured for flushing a second dental tubing that includes a waterline, and wherein the receptacle and the instrument holder are movable, and wherein the instrument holder is separable from the receptacle for use in a freestanding configuration.

14. The flushing device of claim 13, wherein the first flush position defines a generally horizontal insertion axis for the first dental tubing, and wherein the first flush position is configured to cradle the first dental tubing with a distal end of the first dental tubing cantilevered over the receptacle.

15. The flushing device of claim 14, wherein the first flush position is configured to support a tapered surface of the first dental tubing without contacting a distal end of the first dental tubing.

16. The flushing device of claim 14, wherein the first flush position is configured to contact and support a tapered handpiece nut of the first dental tubing.

17. The flushing device of claim 16, wherein the first flush position is sized to accommodate a dental tubing with a connected device or a portion thereof extending distally of the tapered handpiece nut.

18. The flushing device of claim 13, wherein the second flush position defines a generally vertical insertion axis for the second dental tubing, and wherein the second flush position comprises a closely-fitting recess configured to receive a dental syringe body connected to the second dental tubing.

19. The flushing device of claim 18, wherein the second flush position comprises a protrusion in the recess configured to depress a water button of the dental syringe body received in the second flush position.

20. The flushing device of claim 13, wherein the instrument holder and the receptacle are engageable with each other in a nested arrangement.
